(19) United States
(12) Patent Application Publication
(10) Pub. No.: US 2026/0148375 A1
(43) Pub. Date: May 28, 2026

(54) UNIVERSAL FOUNDATION MODEL FRAMEWORK FOR MEDICAL IMAGING

(71) Applicant: Siemens Healthineers AG, Forchheim (DE)

(72) Inventors: Gengyan Zhao, Robbinsville, NJ (US); Badhan Kumar Das, Erlangen (DE); Boris Mailhe, Plainsboro, NJ (US); Han Liu, Nashville, TN (US); Bogdan Georgescu, Princeton, NJ (US); Youngjin Yoo, Princeton, NJ (US); Eli Gibson, Lawrenceville, NJ (US); Dorin Comaniciu, Princeton, NJ (US)

(21) Appl. No.: 18/961,707

(22) Filed: Nov. 27, 2024

(51) Int. Cl. G06T 7/00; G06V 10/40; G06V 10/774; G16H 30/40

(52) U.S. Cl. CPC G06T 7/0012; G06V 10/40; G06V 10/774; G16H 30/40

(57) ABSTRACT

Systems and methods for performing a medical imaging analysis task using a universal foundation model are provided. 1) one or more input medical images each in a domain and 2) a domain code for each of the one or more input medical images identifying its domain are provided. For each respective one of the domain codes, one or more weights are determined based on the respective domain code. One or more parameters of a dynamic convolutional layer are updated based on the one or more weights. A first set of features is extracted from the one or more input medical images using the dynamic convolutional layer with the one or more updated parameters. The first set of features are encoded into a second set of features using a machine learning based encoder. A medical imaging analysis task is performed based on the second set of features. Results of the medical imaging analysis task are output.

### TECHNICAL FIELD

The present invention relates generally to AI/ML (artificial intelligence/machine learning) based medical imaging analysis, and in particular to a universal foundation model framework for medical imaging.

### BACKGROUND

Recently, the advancement of foundation models has garnered significant attention, with notable progress observed in various applications. For instance, LLM (large language model) advancement has significantly improved question answering tasks, while diffusion models have excelled in image synthesis. However, the inherent complexity and heterogeneity of medical imaging data present significant challenges in designing a universal foundation model.

Conventional foundation models typically accept a single image as input. However, medical diagnoses often necessitate the utilization of multiple input medical images, such as multiple MR (magnetic resonance) images with different contrasts. Moreover, the number of input images and the corresponding contrasts acquired may vary across different diagnostic tasks and clinical sites. Conventional foundation models are typically unable to handle varying number of input images.

### BRIEF SUMMARY OF THE INVENTION

In accordance with one or more embodiments, systems and methods for performing a medical imaging analysis task using a universal foundation model are provided. 1) one or more input medical images each in a domain and 2) a domain code for each of the one or more input medical images identifying its domain are provided. For each respective one of the domain codes, one or more weights are determined based on the respective domain code. One or more parameters of a dynamic convolutional layer are updated based on the one or more weights. A first set of features is extracted from the one or more input medical images using the dynamic convolutional layer with the one or more updated parameters. The first set of features are encoded into a second set of features using a machine learning based encoder. A medical imaging analysis task is performed based on the second set of features. Results of the medical imaging analysis task are output.

In one embodiment, the dynamic convolutional layer and the machine learning based encoder are trained by receiving 1) one or more training medical images each in a domain and 2) a training domain code for each of the one or more training medical images identifying its domain. Patches of the one or more training medical images are masked. For each respective one of the training domain codes, one or more additional weights are determined based on the respective training domain code. One or more additional parameters of the dynamic convolutional layer are updated based on the one or more additional weights. A third set of features are extracted from the unmasked patches of the one or more training medical images using the dynamic convolutional layer with the one or more updated additional parameters. The third set of features is encoded into a fourth set of features using the machine learning based encoder. Tokens representing the masked patches of the one or more training medical images are inserted into the fourth set of features. The one or more training medical images are reconstructed based on the fourth set of features with the inserted tokens using a machine learning based decoder. The dynamic convolutional layer and the machine learning based encoder are trained based on the one or more training medical images and the one or more reconstructed training medical images. The trained dynamic convolutional layer and the trained machine learning based encoder are output.

In one embodiment, the respective domain code is projected to the one or more weights using a linear projector.

In one embodiment, a weight parameter and a bias parameter of the dynamic convolutional layer are updated. The one or more parameters of the dynamic convolutional layer are updated by determining a dot product of the one or more parameters of the dynamic convolutional layer and a respective one of the one or more weights.

In one embodiment, the first set of features are encoded with positional embeddings. In another embodiment, modality embeddings are generated based on the domain codes and the first set of features is encoded with the modality embeddings.

In one embodiment, the medical imaging analysis task comprises medical image synthesis.

In accordance with one or more embodiments, systems and methods for training a universal foundation model for performing a medical imaging analysis task are provided. 1) one or more training medical images each in a domain and 2) a domain code for each of the one or more training medical images identifying its domain are received. Patches of the one or more training medical images are masked. For each respective one of the domain codes, one or more weights are determined based on the respective domain code. One or more parameters of a dynamic convolutional layer are updated based on the one or more additional weights. A first set of features are extracted from the unmasked patches of the one or more training medical images using the dynamic convolutional layer with the one or more updated parameters. The first set of features are encoded into a second set of features using a machine learning based encoder. Tokens representing the masked patches of the one or more training medical images are inserted into the second set of features. The one or more training medical images are reconstructed based on the second set of features with the inserted tokens using a machine learning based decoder. The dynamic convolutional layer and the machine learning based encoder are trained based on the one or more training medical images and the one or more reconstructed training medical images. The trained dynamic convolutional layer and the trained machine learning based encoder are output.

These and other advantages of the invention will be apparent to those of ordinary skill in the art by reference to the following detailed description and the accompanying drawings.

### BRIEF DESCRIPTION OF THE DRAWINGS

FIG. 1 shows a method for performing a medical imaging analysis task using a universal foundation model, in accordance with one or more embodiments;

FIG. 2 shows a workflow for performing a medical imaging analysis task using a universal foundation model, in accordance with one or more embodiments;

FIG. 3 shows a method for training a universal foundation model for performing a medical imaging analysis task, in accordance with one or more embodiments;

FIG. 4 shows a workflow for training a universal foundation model for performing a medical imaging analysis task, in accordance with one or more embodiments;

FIG. 5 shows a table comparing performance of the AMAE in accordance with embodiments described herein with conventional approaches;

FIG. 6 shows a table comparing the AMAE in accordance with embodiments described herein with conventional approaches where AxT2 is missing for all subjects;

FIG. 7 shows a table statistically comparing the AMAE in accordance with embodiments described herein with conventional approaches with and without AMAE pretrained weights;

FIG. 8 shows an exemplary artificial neural network that may be used to implement one or more embodiments;

FIG. 9 shows a convolutional neural network that may be used to implement one or more embodiments;

FIG. 10 shows a data flow diagram of a generative adversarial network that may be used to implement one or more embodiments;

FIG. 11 shows a schematic structure of a recurrent machine learning model that may be used to implement one or more embodiments; and

FIG. 12 shows a high-level block diagram of a computer that may be used to implement one or more embodiments.

### DETAILED DESCRIPTION

The present invention generally relates to methods and systems for a universal foundation model framework for medical imaging. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a novel universal foundation model framework for efficiently handling varying number of input medical images. The framework eliminates the need for training multiple models or encoders to accommodate varying numbers of input images. The foundation model in accordance with embodiments described herein can take advantage of all available input data with minimal preprocessing, and the pretrained foundation model can be leveraged to address various downstream tasks with disparate requirements and varying numbers of input medical images.

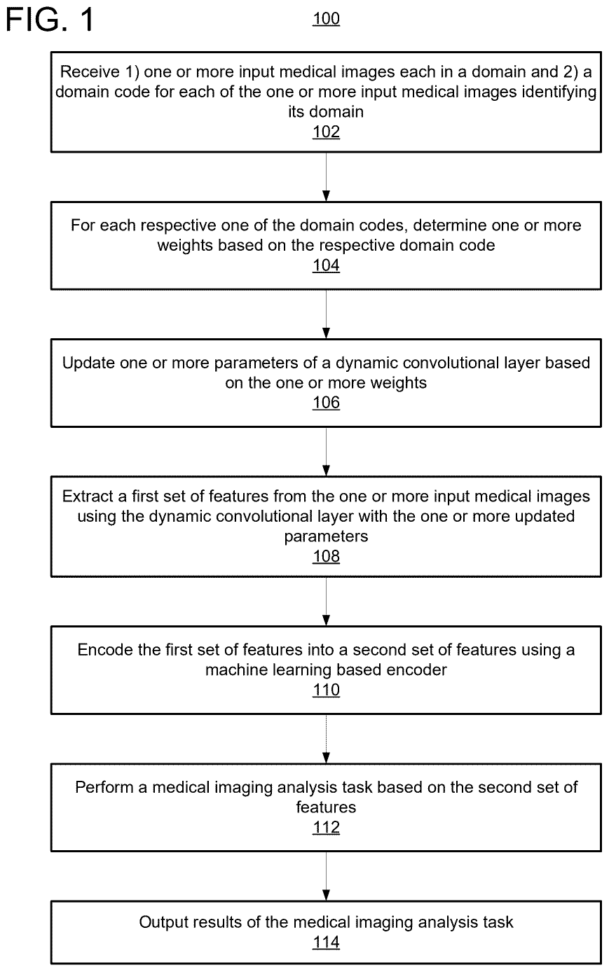

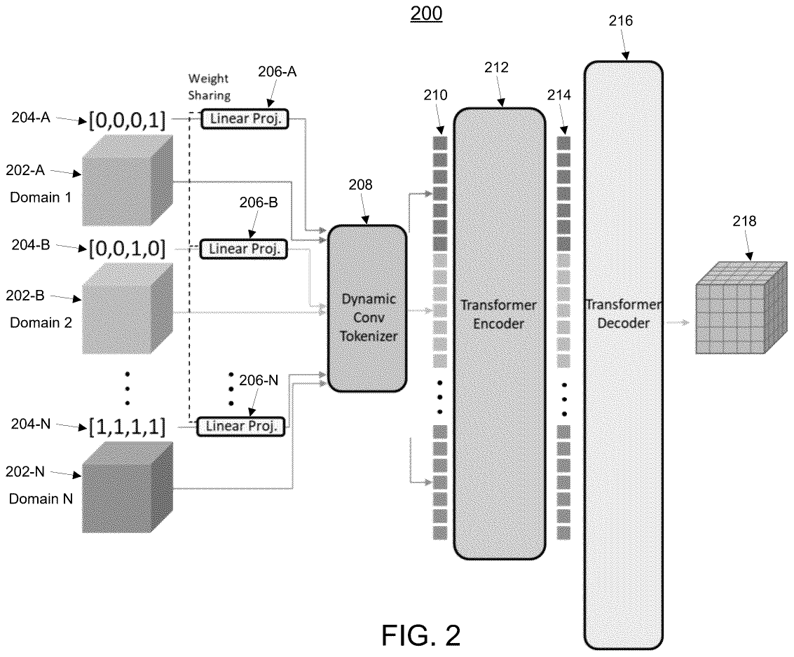

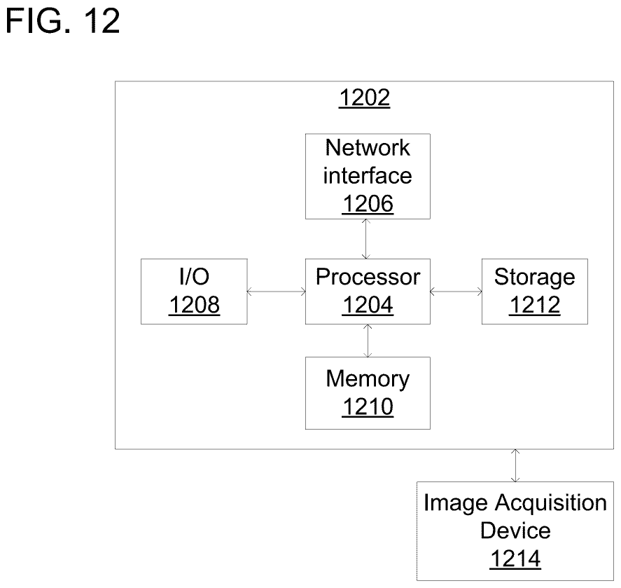

FIG. 1 shows a method 100 for performing a medical imaging analysis task using a universal foundation model, in accordance with one or more embodiments. The steps and sub-steps of method 100 may be performed by one or more suitable computing devices, such as, e.g., computer 1202 of FIG. 12. FIG. 2 shows a workflow 200 for performing a medical imaging analysis task using a universal foundation model, in accordance with one or more embodiments. FIG. 1 and FIG. 2 will be described together.

At step 102 of FIG. 1, 1) one or more input medical images each in a domain and 2) a domain code for each of the one or more input medical images identifying its domain are received. As shown in workflow 200 of FIG. 2, the one or more input medical images may be input medical images 202-A, 202-B, . . . , 202-N (collectively referred to as input medical images 202) respectively in domains 1, 2, . . . , N and the domain codes may be domain codes 204-A, 204-B, . . . , 204-N (collectively referred to as domain codes 204) respectively identifying the domain of input medical images 202-A, 202-B, . . . , 202-N. In particular, domain code 204-A is [0,0,0,1] identifying domain 1, domain code 204-B is [0,0,1,0] identifying domain 2, and domain code 204-N is [1,1,1,1] identifying domain N.

The one or more input medical images may depict any anatomical object of interest of a patient and may be of any suitable domain. As used herein, a domain of a medical image refers to the modality of the medical image as well as the protocol used for obtaining the medical image in that modality. The modality of the one or more input medical images may include, for example, MRI (magnetic resonance imaging), CT (computed tomography), US (ultrasound), x-ray, SPECT (single-photon emission computed tomography), PET (positron emission tomography), or any other medical imaging modality or combinations of medical imaging modalities. The protocol used for obtaining the medical image may include, for example, acquisition sequences or techniques for acquiring a medical image, such as, e.g., T1-weighted, T2-weighted, proton density-weighted MRI images, contrast and non-contrast images, CT images captured with low kV (kilovoltage) and high kV, or low and high resolution medical images. Accordingly, the domains may be completely different medical imaging modalities or different image protocols within the same overall imaging modality. The one or more input medical images may be represented in the image space (e.g., as pixel or voxel values in spatial coordinates) or the latent space (e.g., as a lower-dimensional, compressed representation of the one or more medical images represented as a feature vector). The one or more input medical images in the image space may be 2D (two dimensional) images and/or 3D (three dimensional) volumes.

The domain codes respectively identify the domain of the one or more input medical images. The domain codes may be represented in any suitable form. In one embodiment, the domain codes are vectors. Each combination of values of the domain codes are associated with a predefined domain. The association between the domain codes and the predefined domains may be predefined by a user or learned during training. Other approaches for encoding the identification of a domain in a domain code are also contemplated.

The one or more input medical images and/or the domain codes may be received, for example, by directly receiving the one or more input medical images from an image acquisition device (e.g., image acquisition device 1214 of FIG. 12) as the one or more input medical images are acquired, by loading the one or more input medical images and/or the domain codes from a storage or memory of a computer system (e.g., storage 1212 or memory 1210 of computer 1202 of FIG. 12), or by receiving the one or more input medical images and/or the domain codes from a remote computer system (e.g., computer 1202 of FIG. 12). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 104 of FIG. 1, for each respective one of the domain codes, one or more weights are determined based on the respective domain code. The one or more weights are for updating one or more parameters of a dynamic convolutional layer (updated at step 106 of FIG. 1). In one embodiment, the one or more weights comprise a weight vector w_{conv} and a bias vector b_{conv} for respectively updating the weight parameter W and the bias parameter of B the dynamic convolutional layer.

The one or more weights may be determined using any suitable approach. In one embodiment, the one or more weights are determined using a learnable linear projector. In one example, as shown in workflow 200 of FIG. 2, one or more weights are determined by each linear projector 206-A, 206-B, . . . , 206-N (collectively referred to as linear projectors 206) based on domain codes 204-A, 204-B, . . . , 204-N, respectively. A linear projector is a linear transformation learned during the training process. The linear projector projects or maps a domain code to the one or more weights through a set of parameters optimized during the training process. While linear projectors 206-A, 206-B, . . . , 206-N are separately shown in workflow 200 to illustrate the linear projection of domain codes 204-A, 204-B, . . . , 204-N to one or more weights, it should be understood that linear projectors 206-A, 206-B, . . . , 206-N share the same weights and thus represent the same linear projector 206.

At step 106 of FIG. 1, one or more parameters of a dynamic convolutional layer are updated based on the one or more weights. In one example, as shown in workflow 200 of FIG. 2, one or more parameters of dynamic convolutional layer/tokenizer 208 are updated based on the one or more weights output by linear projectors 206.

The dynamic convolutional layer comprises convolutional filters (or kernels) that are not fixed but adapt dynamically by updating the one or more parameters based on the domain codes. Examples of a dynamic convolutional layer include dynamic filter networks and attention mechanisms. In one embodiment, the one or more parameters of the dynamic convolutional layer comprise a weight parameter W and a bias parameter B. In one embodiment, the one or more parameters are updated by calculating the dot product. For example, the weight parameter may be updated by calculating the dot product of the weight parameter W and the weight vector w_{conv} (i.e., W_{updated}=W·w_{conv}) and the bias parameter may be updated by calculating the dot product of the bias parameter B and the bias vector b_{conv} (i.e., B_{updated}=B·b_{conv}). Thus, the domain codes control the behavior of the dynamic convolutional layer for each domain to extract corresponding features. The one or more parameters may be updated according to any other suitable approach.

At step 108 of FIG. 1, a first set of features is extracted from the one or more input medical images using the dynamic convolutional layer with the one or more updated parameters. In one example, as shown in workflow 200 of FIG. 2, first set of features 210 are extracted from medical images 202 using dynamic convolutional layer/tokenizer 208. The dynamic convolutional layer receives as input patches extracted from the one or more input medical images, maps each patch to a token of features, and generates as output the tokens representing the first set of features. The features are a lower-dimensional, compressed representation of the one or more input medical images represented as a feature vector.

The dynamic convolutional layer allows the extraction of domain-specific features from each of the one or more input medical images, while also creating non-overlapping tokens for the machine learning based encoder (utilized at step 110 of FIG. 1) by using the patch size as the kernel and stride size of the convolution and by using the output dimension of the convolution as the embedding dimension of the machine learning based encoder. Let Xᵢ∈ [symbol] represent one of the one or more input medical images (or image tensor), where H, W, and D are the height, width, and depth of the input medical image respectively. The convolution operation with the updated weight parameter and bias parameter is:

$${Y}_{i} = C o n v ( X , {W}_{updated} , {B}_{updated} )$$

where

$${Y}_{i} ∈ {( \frac{H \times W \times D}{{patchsize}^{3}} ) \times EmbeddingDim}^{}$$

is the output tokens produced by the dynamic convolutional layer for one input medical image.

At step 110 of FIG. 1, the first set of features is encoded into a second set of features using a machine learning based encoder. In one example, as shown in workflow 200 of FIG. 2, first set of features 210 are encoded into second set of features 214 by transformer encoder 212.

The tokens of the first set of features are combined (e.g., concatenated) into a single sequence and input into the machine learning based encoder. The tokens of the first set of features are encoded with positional embeddings (e.g., sinusoidal or learnable) based on the position of their patch within the one or more input medical images. Thus, tokens of different domains but the same relative patch location will have the same positional embedding. The position embeddings help preserve spatial relationships of the tokens. In addition, modality embeddings are generated from the domain codes using another linear projector and the tokens of the first set of features are encoded with modality embeddings. The machine learning based encoder receives as input the first set of features (represented as a sequence of tokens encoded with the position embeddings and the modality embeddings) and generates as output the second set of features. In one embodiment, the machine learning based encoder is a transformer encoder. However, the machine learning based encoder may be implemented in any other suitable form.

At step 112 of FIG. 1, a medical imaging analysis task is performed based on the second set of features. The medical imaging analysis task may be performed using a machine learning based task network, such as, e.g., a task-specific decoder head (e.g., a transformer decoder). In one example, as shown in workflow 200 of FIG. 2, transformer decoder 216 generates synthetic medical images 218 based on second set of features 214. The machine learning based task network receives as input the second set of features and generates as output results of the medical imaging analysis task. In one embodiment, the medical imaging analysis task is image synthesis for generating a synthetic image from the one or more medical images. The synthetic image may be in a domain that is absent from the domains of the one or more input medical images. The medical imaging analysis task may additionally or alternatively comprise any other suitable task, such as, e.g., detection, segmentation, classification, quantification, etc.

At step 114 of FIG. 1, results of the medical imaging analysis task are output. For example, the results of the medical imaging analysis task can be output by displaying the results on a display device of a computer system (e.g., I/O 1208 of computer 1202 of FIG. 12), storing the results on a memory or storage of a computer system (e.g., memory 1210 or storage 1212 of computer 1202 of FIG. 12), or by transmitting the results to a remote computer system (e.g., computer 1202 of FIG. 12).

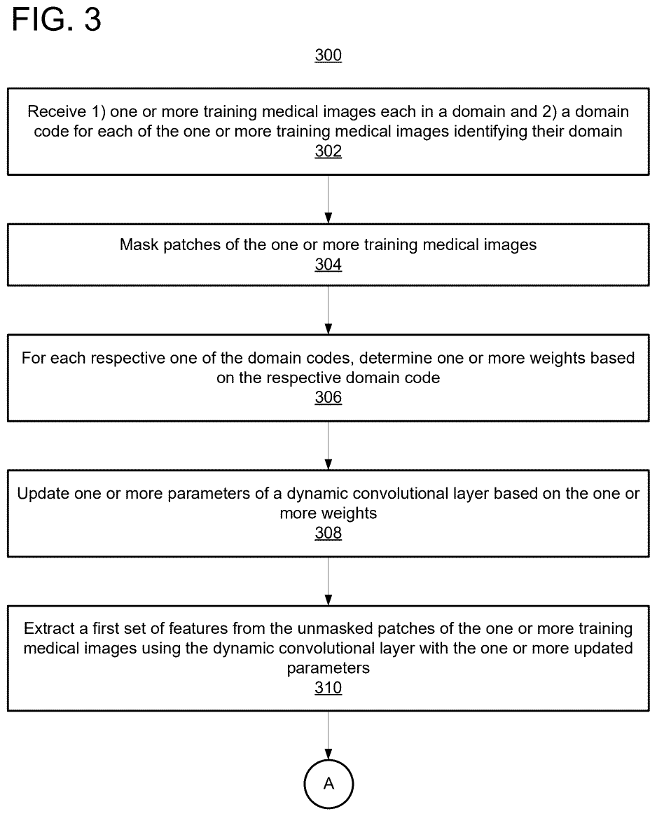

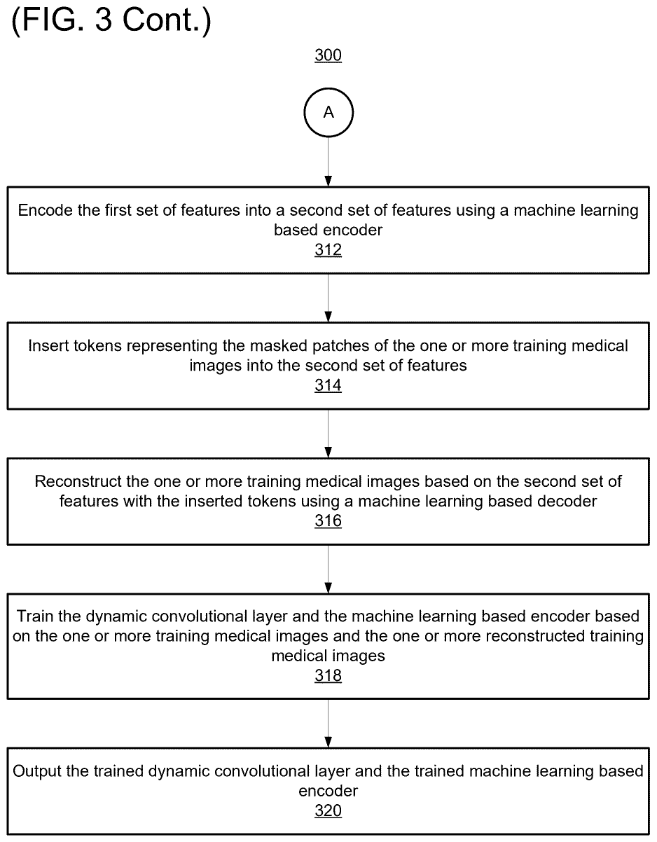

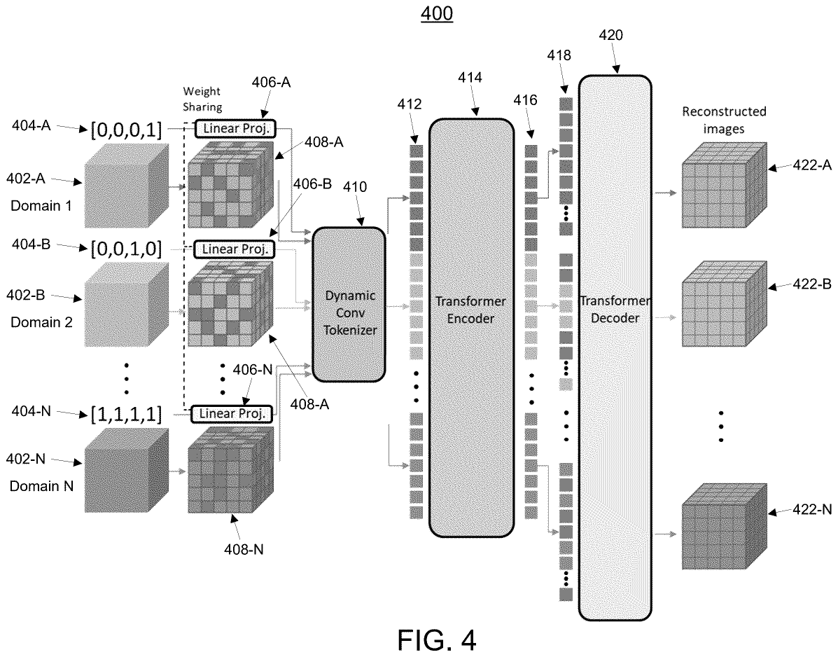

The dynamic convolutional layer and the machine learning based encoder are trained during a prior offline or training stage, e.g., according to method 300 of FIG. 3 and/or workflow 400 of FIG. 4. Once trained, the dynamic convolutional layer and the machine learning based encoder are applied during an online or inference stage, e.g., to perform steps 108 and 110 of FIG. 1.

FIG. 3 shows a method 300 for training a universal foundation model for performing a medical imaging analysis task, in accordance with one or more embodiments. The steps and sub-steps of method 300 may be performed by one or more suitable computing devices, such as, e.g., computer 1202 of FIG. 12. FIG. 4 shows a workflow 400 for training a universal foundation model for performing a medical imaging analysis task, in accordance with one or more embodiments. FIG. 3 and FIG. 4 will be described together. The steps of method 300 of FIG. 3 and workflow 400 of FIG. 4 are performed during an offline or training stage for training a dynamic convolutional layer and a machine learning based encoder.

At step 302 of FIG. 3, 1) one or more training medical images each in a domain and 2) a domain code for each of the one or more training medical images identifying its domain are received. As shown in workflow 400 of FIG. 4, the one or more training medical images may be training medical images 402-A, 402-B, . . . , 402-N (collectively referred to as training medical images 402) respectively in domains 1, 2, . . . , N and the domain codes may be domain codes 404-A, 404-B, . . . , 404-N (collectively referred to as domain codes 404) respectively identifying the domain of training medical images 402-A, 402-B, . . . , 402-N. In particular, domain code 404-A is [0,0,0,1] identifying domain 1, domain code 404-B is [0,0,1,0] identifying domain 2, and domain code 404-N is [1,1,1,1] identifying domain N.

The one or more training medical images may be of any suitable domain. The one or more training medical images may be represented in the image space or the latent space. The one or more training medical images in the image space may be 2D images and/or 3D volumes.

The domain codes respectively identify the domain of the one or more training medical images. The domain codes may be represented in any suitable form (e.g., vectors). Each combination of values of the domain codes are associated with a predefined domain (e.g., by a user or learned during training). Other approaches for encoding the identification of a domain in a domain code are also contemplated.

The one or more training medical images and/or the domain codes may be received, for example, by directly receiving the one or more training medical images from an image acquisition device (e.g., image acquisition device 1214 of FIG. 12) as the one or more training medical images are acquired, by loading the one or more training medical images and/or the domain codes from a storage or memory of a computer system (e.g., storage 1212 or memory 1210 of computer 1202 of FIG. 12), or by receiving the one or more training medical images and/or the domain codes from a remote computer system (e.g., computer 1202 of FIG. 12).

At step 304 of FIG. 3, patches of the one or more training medical images are masked. In one example, as shown in workflow 400 of FIG. 4, patches of training medical images 402-A, 402-B, . . . , 402-N are respectively masked to provide masked training medical images 406-A, 406-B, . . . , 406-N. The patches of the one or more training medical images may be masked according to any suitable approach (e.g., randomly).

At step 306 of FIG. 3, for each respective one of the domain codes, one or more weights are determined based on the respective domain code. The one or more weights are for updating one or more parameters of a dynamic convolutional layer (updated at step 308 of FIG. 3). In one embodiment, the one or more weights comprise a weight vector w_{conv} and a bias vector b_{conv} for updating the weight parameter W and the bias parameter of B the dynamic convolutional layer.

The one or more weights may be determined using any suitable approach. In one embodiment, the one or more weights are determined using a learnable linear projector. In one example, as shown in workflow 400 of FIG. 4, one or more weights are determined by each linear projector 406-A, 406-B, . . . , 406-N (collectively referred to as linear projectors 406) based on domain codes 404-A, 404-B, . . . , 404-N, respectively. While linear projectors 406-A, 406-B, . . . , 406-N are separately shown in workflow 400 to illustrate the linear projection of domain codes 404-A, 404-B, . . . , 404-N to one or more weights, it should be understood that linear projectors 406-A, 406-B, . . . , 406-N share the same weights and thus represent the same linear projector 406.

At step 308 of FIG. 3, one or more parameters of a dynamic convolutional layer are updated based on the one or more weights. In one example, as shown in workflow 400 of FIG. 4, one or more parameters of dynamic convolutional layer/tokenizer 410 are updated based on the one or more weights output by linear projectors 406. The dynamic convolutional layer may be the dynamic convolutional layer utilized at step 108 of FIG. 1 or may be dynamic convolutional layer/tokenizer 208 of FIG. 2.

In one embodiment, the one or more parameters of the dynamic convolutional layer comprise a weight parameter W and a bias parameter B. In one embodiment the one or more parameters are updated by calculating the dot product. For example, the weight parameter may be updated by calculating the dot product of the weight parameter W and the weight vector w_{conv} (i.e., W_{updated}=W·w_{conv}) and the bias parameter may be updated by calculating the dot product of the bias parameter B and the bias vector b_{conv} (i.e., B_{updated}=B·b_{conv}). The one or more parameters may be updated according to any other suitable approach.

At step 310 of FIG. 3, a first set of features is extracted from the unmasked patches of the one or more training medical images using the dynamic convolutional layer with the one or more updated parameters. In one example, as shown in workflow 400 of FIG. 4, first set of features 412 are extracted from unmasked patches of training medical images 402 using dynamic convolutional layer/tokenizer 410. The dynamic convolutional layer receives as input the unmasked patches extracted from the one or more training medical images, maps each patch to a token of features, and generates as output the tokens representing the first set of features. The features are a lower-dimensional, compressed representation of the one or more input medical images represented as a feature vector.

At step 312 of FIG. 3, the first set of features is encoded into a second set of features using a machine learning based encoder. In one example, as shown in workflow 400 of FIG. 4, first set of features 412 are encoded into second set of features 416 by transformer encoder 414. The machine learning based encoder may be the machine learning based encoder utilized at step 108 of FIG. 1 or may be transformer encoder 212 of FIG. 2.

The tokens of the first set of features are combined (e.g., concatenated) into a single sequence and input into the machine learning based encoder. The tokens of the first set of features are encoded with positional embeddings based on the position of their patch within the one or more input medical images. In addition, modality embeddings are generated from the domain codes using another linear projector and the tokens of the first set of features are encoded with modality embeddings. The machine learning based encoder receives as input the first set of features, represented as a sequence of tokens encoded with the position embeddings and the modality embeddings, and generates as output the second set of features. In one embodiment, the machine learning based encoder is a transformer encoder. However, the machine learning based encoder may be implemented in any other suitable form.

At step 314 of FIG. 3, tokens representing the masked patches of the one or more training medical images are inserted into the second set of features. In one example, as shown in workflow 400 of FIG. 4, tokens representing the masked patches are inserted into the second set of features 416 to provide features 418. The tokens representing the masked patches are placeholder tokens inserted into the sequence of the second set of features at the position of each masked patch.

At step 316 of FIG. 3, the one or more training medical images are reconstructed based on the second set of features with the inserted tokens using a machine learning based decoder. In one example, as shown in workflow 400 of FIG. 4, training medical images 402-A, 402-B, . . . , 402-N are reconstructed based on features 418 by transformer decoder 420 to generate reconstructed images 422-A, 422-B, . . . , 422-N.

In one embodiment, the machine learning based decoder is a transformer decoder. However, the machine learning based decoder may be implemented according to any other suitable architecture. The machine learning based decoder receives as input the second set of features with the inserted tokens and generates as output the reconstructed images. In one embodiment, the decoding may be performed by self-attention of the transformer decoder. In this embodiment, the second set of features with the inserted tokens are decoded with self-attention, and then a linear projector is used to map the decoded tokens to the size of the original patches. In another embodiment, the decoding may be performed by cross-attention. In this embodiment, the second set of features with the inserted tokens of a particular domain are used to generate a query, cross-attention is performed with keys and values generated from all encoded tokens from all domains, and a linear projector is used to map the decoded tokens to the size of the original patches. Both self-attention and cross-attention utilize the positional embeddings and the modality embeddings.

At step 318 of FIG. 3, the dynamic convolutional layer and the machine learning based encoder are trained based on the one or more training medical images and the one or more reconstructed training medical images. In one embodiment, the dynamic convolutional layer and the machine learning based encoder are trained by comparing the one or more training medical images and the one or more reconstructed training medical images according to a loss function. In one embodiment, the loss function is an L2 loss function. However, the loss function may comprise any other suitable loss function.

At step 320 of FIG. 3, the trained dynamic convolutional layer and the trained machine learning based encoder are output. For example, the trained dynamic convolutional layer and the trained machine learning based encoder can be output by storing the trained dynamic convolutional layer and the trained machine learning based encoder on a memory or storage of a computer system (e.g., memory 1210 or storage 1212 of computer 1202 of FIG. 12) or by transmitting the trained dynamic convolutional layer and the trained machine learning based encoder to a remote computer system (e.g., computer 1202 of FIG. 12). The trained dynamic convolutional layer and the trained machine learning based encoder may be applied during an online or inference stage, e.g., to perform method 100 of FIG. 1 or workflow 200 of FIG. 2.

In one embodiment, the transformer encoder and the transformer decoders utilized herein may be replaced with state space models to reduce computational cost.

Embodiments described herein were experimentally validated. For pretraining, 45,374 MR studies were used. For each case, a variable number of MRI contrasts acquired were used, including a set of seven contrasts: Apparent Diffusion Coefficient (ADC), Trace-weighted, T2-weighted, Gradient Echo (GRE), Susceptibility-Weighted, T1-weighted, and Fluid-Attenuated Inversion Recovery (FLAIR), or a subset of these when available. For finetuning, a subset with 1648 training, 193 validation and 215 test subjects were used where acute/subacute brain infarct regions were manually segmented.

The manual segmentation of acute and subacute infarct lesions was performed on Axial Trace-weighted (AxTrace) contrast image series by a radiologist (T. J. R., 10 years of experience). The AxTrace image series and corresponding ADC image map were reviewed by the radiologist. Areas, within the brain parenchyma, of hyperintensity in the TraceW image series with hypo or iso-intensity in the ADC map were considered positive for recent (acute to subacute) infarct by the radiologist and delineated as such in an image mask. The inclusion criteria were studies where at least AxTrace and AxADC contrasts are present and where the radiologist identified at least one infarct lesion. Additionally, Axial T2-weighted contrast is used if it is available.

For finetuning for the task of infarct segmentation, two approaches were used. For the first approach, referred to as Adaptive UNETR (UNet Transformers), the DCT and Transformer encoder were kept as it is and the Transformer decoder was removed. A UNETR based segmentation decoder head was then used. As the number of inputs are variable for each subject, the number of tokens also vary. An adaptive max pooling layer is used to extract the necessary information across all the domains at each level in the UNETR decoder head to transfer variable number of tokens to tensors with fixed sizes to generate the segmentation masks.

Let X denote the output of the transformer encoder:

$$X ∈ {B \times N \times ( \frac{H \times W \times D}{{patchsize}^{3}} ) \times EmbeddingDim}^{}$$

where B is the batch size, N is the number of 3D input images,

$$( \frac{H \times W \times D}{{patchsize}^{3}} )$$

is the number of patches/tokens, and EmbeddingDim is the transformer dimension.

An adaptive max pooling layer was used to extract information from all tokens, resulting in:

$${X}_{n e w} ∈ {B \times ( \frac{H \times W \times D}{{patchsize}^{3}} ) \times EmbeddingDim}^{}$$

In another approach, the original UNETR model was used as it is. All the images from different modalities are concatenated along the channel dimension. Zero-filled tensors are used if any input modality is missing. The weights of the pretrained ViT encoder are transferred from pretraining to the UNETR encoder.

The initial learning rate for self pre-training is 1e-5 and weight decay 0.05. An L2 loss function and weighted Adam optimizer were used. For pre-training, the variable masked autoencoder model was run for 500 epochs with a masking ratio of 70% and batch size of 4.

For finetuning for the task of infarct segmentation, the finetune architecture was run 200 epochs and the best validation result model was saved. A test dataset was then used to compare the performance of different models. The performance of the model in accordance with embodiments described herein was compared with UNet and UNETR. The learning rate for finetuning was 0.0001 with learning rate scheduler and weight decay of 0.05. Diceloss and weighted ADAM optimizer were used for training. Additionally, a test was performed where model performance was evaluated on the test data by omitting the optional contrast AxT2, to observe how the models behave when a contrast/modality is missing.

Dice similarity coefficient was used for quantitative evaluations on the test data. Two-sided pairwise Wilcoxon signed rank test was used to compare the Dice scores of two models with and without pre-training.

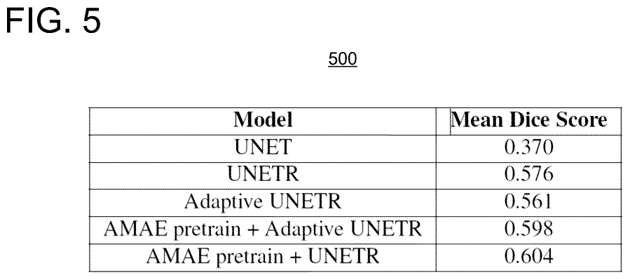

The effectiveness of the proposed AMAE (adaptive masked autoencoder), in accordance with embodiments described herein, pretrained with adaptive UNETR and the original UNETR, are presented in table 500 of FIG. 5. The adaptive UNETR with pretrained weights performed 3.7% better than the adaptive UNETR without the AMAE pretrained weights. Also, in case of the original UNETR, the performance improved by 2.8% after using weights from AMAE pretraining.

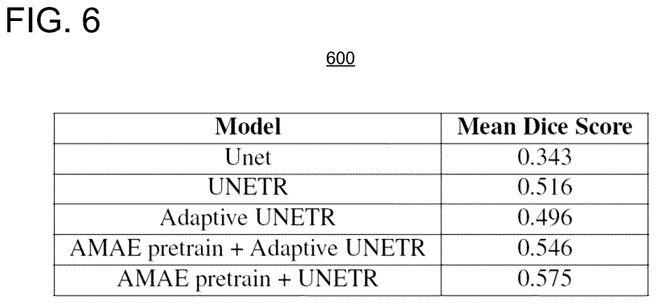

The results in table 600 of FIG. 6 demonstrate the performance of different models on test subjects using the mean Dice score when the optional contrast AxT2 is absent. AMAE pretrained weights with the original UNETR decoder achieved mean Dice score of 0.575 with only 2.9% lower than its performance with all contrasts.

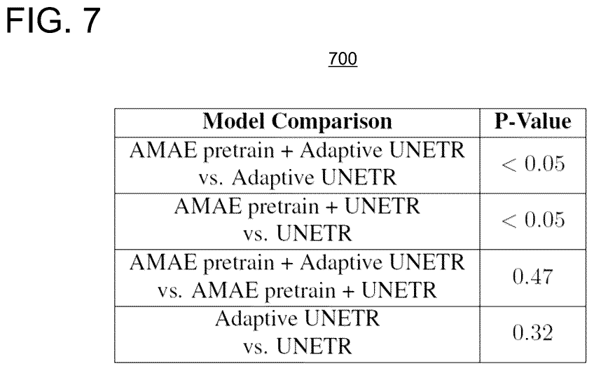

In table 700 of FIG. 7, statistical analysis on Dice score of the two finetune approaches (Adaptive UNETR and UNETR) with pretraining and without pretraining are shown. In both cases, P-value is less than 0.05 which indicate significant difference in segmentation results while applying the pretrained weights compared to the corresponding model without pretrained weights.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the linear projector utilized at step 104, the dynamic convolutional layer utilized at step 108, the machine learning based encoder utilized at step 110, and the machine learning based decoder utilized at step 112 of FIG. 1, linear projectors 206, dynamic convolutional layer 208, transformer encoder 212, and transformer decoder 216 of FIG. 2, the linear projector utilized at step 306, the dynamic convolutional layer utilized at step 310, the machine learning based encoder utilized at step 312, and the machine learning based decoder utilized at step 316 of FIG. 3, and linear projectors 406, dynamic convolutional layer 410, transformer encoder 414, and transformer decoder 420 of FIG. 4, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

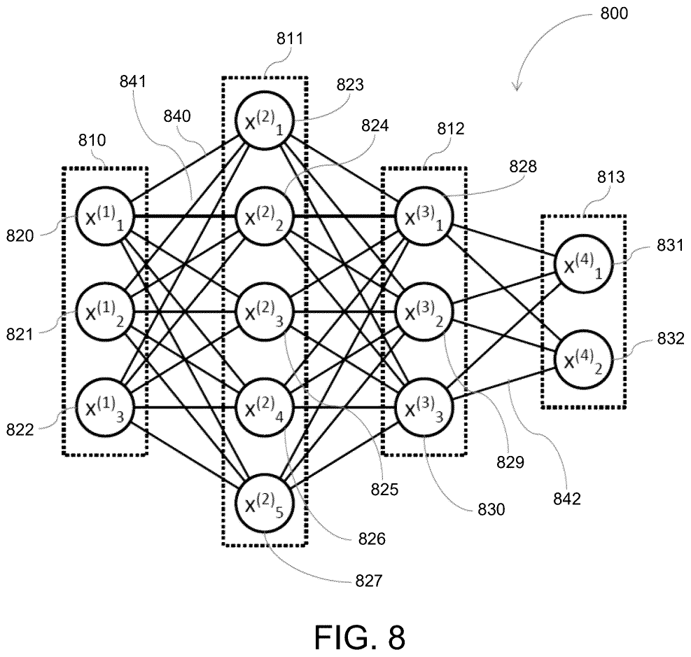

FIG. 8 shows an embodiment of an artificial neural network 800 that may be used to implement one or more machine learning models described herein. Alternative terms for “artificial neural network” are “neural network”, “artificial neural net” or “neural net”.

The artificial neural network 800 comprises nodes 820, . . . , 832 and edges 840, . . . , 842, wherein each edge 840, . . . , 842 is a directed connection from a first node 820, . . . , 832 to a second node 820, . . . , 832. In general, the first node 820, . . . , 832 and the second node 820, . . . , 832 are different nodes 820, . . . , 832, it is also possible that the first node 820, . . . , 832 and the second node 820, . . . , 832 are identical. For example, in FIG. 8 the edge 840 is a directed connection from the node 820 to the node 823, and the edge 842 is a directed connection from the node 830 to the node 832. An edge 840, . . . , 842 from a first node 820, . . . , 832 to a second node 820, . . . , 832 is also denoted as “ingoing edge” for the second node 820, . . . , 832 and as “outgoing edge” for the first node 820, . . . , 832.

In this embodiment, the nodes 820, . . . , 832 of the artificial neural network 800 can be arranged in layers 810, . . . , 813, wherein the layers can comprise an intrinsic order introduced by the edges 840, . . . , 842 between the nodes 820, . . . , 832. In particular, edges 840, . . . , 842 can exist only between neighboring layers of nodes. In the displayed embodiment, there is an input layer 810 comprising only nodes 820, . . . , 822 without an incoming edge, an output layer 813 comprising only nodes 831, 832 without outgoing edges, and hidden layers 811, 812 in-between the input layer 810 and the output layer 813. In general, the number of hidden layers 811, 812 can be chosen arbitrarily. The number of nodes 820, . . . , 822 within the input layer 810 usually relates to the number of input values of the neural network, and the number of nodes 831, 832 within the output layer 813 usually relates to the number of output values of the neural network.

In particular, a (real) number can be assigned as a value to every node 820, . . . , 832 of the neural network 800. Here, x⁽ⁿ⁾ᵢ denotes the value of the i-th node 820, . . . 832 of the n-th layer 810, . . . , 813. The values of the nodes 820, . . . , 822 of the input layer 810 are equivalent to the input values of the neural network 800, the values of the nodes 831, 832 of the output layer 813 are equivalent to the output value of the neural network 800. Furthermore, each edge 840, . . . , 842 can comprise a weight being a real number, in particular, the weight is a real number within the interval [−1, 1] or within the interval [0, 1]. Here, w^{(m,n)}_{i,j} denotes the weight of the edge between the i-th node 820, . . . , 832 of the m-th layer 810, . . . , 813 and the j-th node 820, . . . , 832 of the n-th layer 810, . . . , 813. Furthermore, the abbreviation w⁽ⁿ⁾_{i,j} is defined for the weight w^{(n,n+1)}_{i,j}.

In particular, to calculate the output values of the neural network 800, the input values are propagated through the neural network. In particular, the values of the nodes 820, . . . , 832 of the (n+1)-th layer 810, . . . , 813 can be calculated based on the values of the nodes 820, . . . , 832 of the n-th layer 810, . . . , 813 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 810 are given by the input of the neural network 800, wherein values of the first hid-den layer 811 can be calculated based on the values of the input layer 810 of the neural network, wherein values of the second hidden layer 812 can be calculated based in the values of the first hidden layer 811, etc.

In order to set the values w^{(m,n)}_{i,j} for the edges, the neural network 800 has to be trained using training data. In particular, training data comprises training input data and training output data (denoted as tᵢ). For a training step, the neural network 800 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 800 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 813, wherein f′ is the first derivative of the activation function, and t⁽ⁿ⁺¹⁾ⱼ is the comparison training value for the j-th node of the output layer 813.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

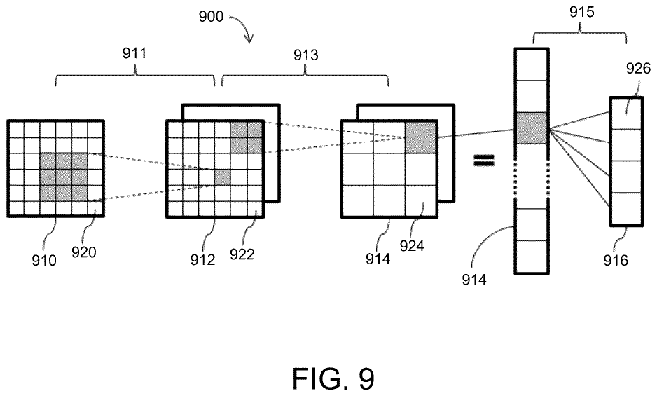

FIG. 9 shows an embodiment of a convolutional neural network 900 that may be used to implement one or more machine learning models described herein. In the displayed embodiment, the convolutional neural network 900 comprises an input node layer 910, a convolutional layer 911, a pooling layer 913, a fully connected layer 914 and an output node layer 916, as well as hidden node layers 912, 914. Alternatively, the convolutional neural network 900 can comprise several convolutional layers 911, several pooling layers 913 and several fully connected layers 915, as well as other types of layers. The order of the layers can be chosen arbitrarily, usually fully connected layers 915 are used as the last layers before the output layer 916.

In particular, within a convolutional neural network 900 nodes 920, 922, 924 of a node layer 910, 912, 914 can be considered to be arranged as a d-dimensional matrix or as a d-dimensional image. In particular, in the two-dimensional case the value of the node 920, 922, 924 indexed with i and j in the n-th node layer 910, 912, 914 can be denoted as x(n)[i, j]. However, the arrangement of the nodes 920, 922, 924 of one node layer 910, 912, 914 does not have an effect on the calculations executed within the convolutional neural network 900 as such, since these are given solely by the structure and the weights of the edges.

A convolutional layer 911 is a connection layer between an anterior node layer 910 (with node values x(n−1)) and a posterior node layer 912 (with node values x(n)). In particular, a convolutional layer 911 is characterized by the structure and the weights of the incoming edges forming a convolution operation based on a certain number of kernels. In particular, the structure and the weights of the edges of the convolutional layer 911 are chosen such that the values x(n) of the nodes 922 of the posterior node layer 912 are calculated as a convolution x(n)=K*x(n−1) based on the values x(n−1) of the nodes 920 anterior node layer 910, where the convolution * is defined in the two-dimensional case as

$${x}_{k}^{( n )} [ i , j ] = ( K {}^{*} {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 920, 922 (e.g., a 3×3 matrix, or a 5×5 matrix). In particular, this implies that the weights of the edges in the convolution layer 911 are not independent, but chosen such that they produce said convolution equation. In particular, for a kernel being a 3×3 matrix, there are only 9 independent weights (each entry of the kernel matrix corresponding to one independent weight), irrespectively of the number of nodes 920, 922 in the anterior node layer 910 and the posterior node layer 912.

In general, convolutional neural networks 900 use node layers 910, 912, 914 with a plurality of channels, in particular, due to the use of a plurality of kernels in convolutional layers 911. In those cases, the node layers can be considered as (d+1)-dimensional matrices (the first dimension indexing the channels). The action of a convolutional layer 911 is then a two-dimensional example defined as

$${x}^{{( n )}_{b}} [ i , j ] = {\sum }_{a} {K}_{a , b} {}^{*} {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾ₐ corresponds to the a-th channel of the anterior node layer 910, x⁽ⁿ⁾_{b} corresponds to the b-th channel of the posterior node layer 912 and K_{a,b} corresponds to one of the kernels. If a convolutional layer 911 acts on an anterior node layer 910 with A channels and outputs a posterior node layer 912 with B channels, there are A·B independent d-dimensional kernels K_{a,b}.

In general, in convolutional neural networks 900 activation functions are used. In this embodiment ReLU (acronym for “Rectified Linear Units”) is used, with R(z)=max(0, z), so that the action of the convolutional layer 911 in the two-dimensional example is

$${x}^{{( n )}_{b}} [ i , j ] = R ( {\sum }_{a} ( {K}_{a , b} {}^{*} {x}^{{( n - 1 )}_{a}} ) [ i , j ] = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 910 comprises 36 nodes 920, arranged as a two-dimensional 6×6 matrix. The first hidden node layer 912 comprises 72 nodes 922, arranged as two two-dimensional 6×6 matrices, each of the two matrices being the result of a convolution of the values of the input layer with a 3×3 kernel within the convolutional layer 911. Equivalently, the nodes 922 of the first hidden node layer 912 can be interpreted as arranged as a three-dimensional 2×6×6 matrix, wherein the first dimension correspond to the channel dimension.

The advantage of using convolutional layers 911 is that spatially local correlation of the input data can exploited by enforcing a local connectivity pattern between nodes of adjacent layers, in particular by each node being connected to only a small region of the nodes of the preceding layer.

A pooling layer 913 is a connection layer between an anterior node layer 912 (with node values x(n−1)) and a posterior node layer 914 (with node values x(n)). In particular, a pooling layer 913 can be characterized by the structure and the weights of the edges and the activation function forming a pooling operation based on a non-linear pooling function f. For example, in the two-dimensional case the values x(n) of the nodes 924 of the posterior node layer 914 can be calculated based on the values x(n−1) of the nodes 922 of the anterior node layer 912 as

$${x}^{{( n )}_{b}} [ i , j ] = f ( {x}^{( n - 1 )} [ {id}_{1 ,} {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 913 the number of nodes 922, 924 can be reduced, by re-placing a number d1·d2 of neighboring nodes 922 in the anterior node layer 912 with a single node 922 in the posterior node layer 914 being calculated as a function of the values of said number of neighboring nodes. In particular, the pooling function f can be the max-function, the average or the L2-Norm. In particular, for a pooling layer 913 the weights of the incoming edges are fixed and are not modified by training.

The advantage of using a pooling layer 913 is that the number of nodes 922, 924 and the number of parameters is reduced. This leads to the amount of computation in the network being reduced and to a control of overfitting.

In the displayed embodiment, the pooling layer 913 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 900 are fully connected layers 915. A fully connected layer 915 is a connection layer between an anterior node layer 914 and a posterior node layer 916. A fully connected layer 913 can be characterized by the fact that a majority, in particular, all edges between nodes 914 of the anterior node layer 914 and the nodes 916 of the posterior node layer are present, and wherein the weight of each of these edges can be adjusted individually.

In this embodiment, the nodes 924 of the anterior node layer 914 of the fully connected layer 915 are displayed both as two-dimensional matrices, and additionally as non-related nodes (indicated as a line of nodes, wherein the number of nodes was reduced for a better presentability). This operation is also denoted as “flattening”. In this embodiment, the number of nodes 926 in the posterior node layer 916 of the fully connected layer 915 smaller than the number of nodes 924 in the anterior node layer 914. Alternatively, the number of nodes 926 can be equal or larger.

Furthermore, in this embodiment the Softmax activation function is used within the fully connected layer 915. By applying the Softmax function, the sum the values of all nodes 926 of the output layer 916 is 1, and all values of all nodes 926 of the output layer 916 are real numbers between 0 and 1. In particular, if using the convolutional neural network 900 for categorizing input data, the values of the output layer 916 can be interpreted as the probability of the input data falling into one of the different categories.

In particular, convolutional neural networks 900 can be trained based on the backpropagation algorithm. For preventing overfitting, methods of regularization can be used, e.g., dropout of nodes 920, . . . , 924, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

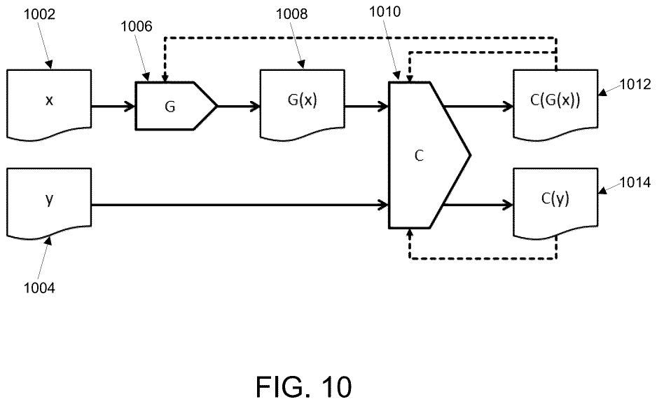

FIG. 10 shows a data flow diagram according to an embodiment for using a generative adversarial network for creating synthetic output data G(x) 1008 based on input data x 1002 that is indistinguishable from real output data y 1004, in accordance with one or more embodiments. The synthetic output data G(x) 1008 has the same structure as the real output data y 1004, but its content is not derived from real world data.

The generative adversarial network comprises a generator function G 1006 and a classifier function C 1010 which are trained jointly. The task of the generator function G 1006 is to provide realistic synthetic output data G(x) 1008 based on input data x 1002, and the task of the classifier function C 1010 is to distinguish between real output data y 1004 and synthetic output data G(x) 1008. In particular, the output of the classifier function C 1010 is a real number between 0 and 1 corresponding to the probability of the input value being real data, so that an ideal classifier function would calculate an output value of C(y) 1014≈1 for real data y 1004 and C(G(x)) 1012≈0 for synthetic data G(x) 1008.

Within the training process, parameters of the generator function G 1006 are adapted so that the synthetic output data G(x) 1008 has the same characteristics as real output data y 1004, so that the classifier function C 1010 cannot distinguish between real and synthetic data anymore. At the same time, parameters of the classifier function C 1010 are adapted so that it distinguishes between real and synthetic data in the best possible way. Here, the training relies on pairs comprising input data x 1002 and the corresponding real output data y 1004. Within a single training step, the generator function G 1006 is applied to the input data x 1002 for generating synthetic output data G(x) 1008. Furthermore, the classifier function C 1010 is applied to the real output data y 1004 for generating a first classification result C(y) 1014. Additionally, the classifier function C 1010 is applied to the synthetic output data G(x) 1008 for generating a second classification result C(G(x)) 1012.

Adapting the parameters of the generative function G 1006 and the classifier function C 1010 is based on minimizing a cost function by using the backpropagation algorithm, respectively. In this embodiment, the cost function K_{C} for the classifier function C 1010 is K_{C}∝−BCE(C(y), 1)−BCE(C(G(x), 0), wherein BCE denotes the binary cross entropy defined as BCE(z, z′)=z′·log(z)+(1−z′)·log(1−z). By using this cost function, both wrongly classifying real output data as synthetic (indicated by C(y)≈0) and wrongly classifying synthetic output data as real (indicated as C(G(x)) 1012≈1) increases the cost function K_{C} to be minimized. Furthermore, the cost function K_{G} for the generator function G 1006 is K_{G}∝−BCE(C(G(x), 1)=−log(C(G(x). By using this cost function, correctly classified synthetic output data (indicated as C(G(x)) 1012≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

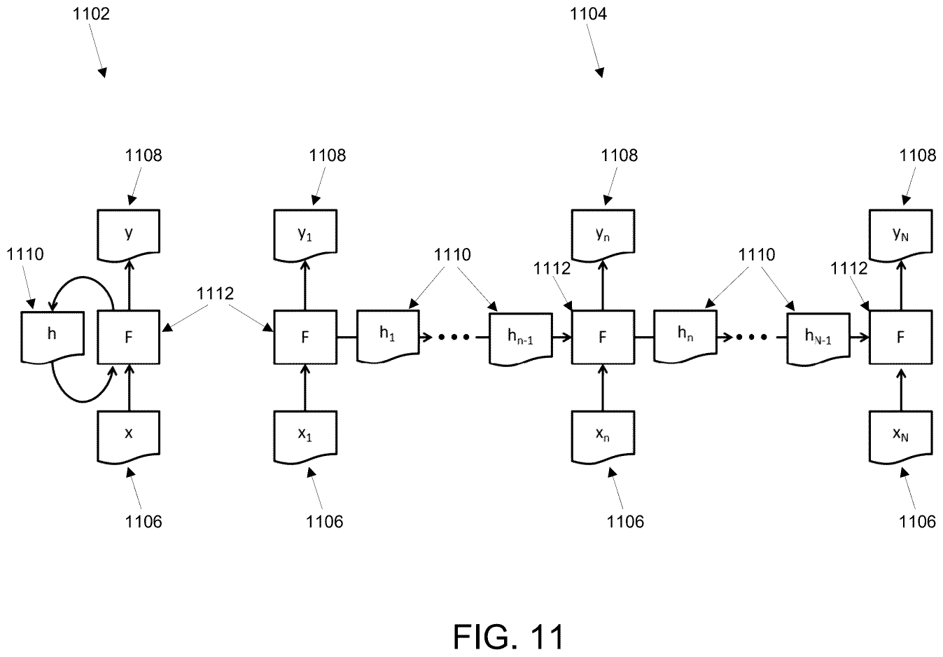

FIG. 11 shows the schematic structure of a recurrent machine learning model F, both in a recurrent representation 1102 and in an unfolded representation 1104, that may be used to implement one or more machine learning models described herein. The recurrent machine learning model takes as input several input datasets x, x₁, . . . , x_{N} 1106 and creates a corresponding set of output datasets y, y₁, . . . , y_{N} 1108. Furthermore, the output depends on a so-called hidden vector h, h₁, . . . , h_{N} 1110, which implicitly comprises information about input datasets previously used as input for the recurrent machine learning model F 1112. By using these hidden vectors h, h₁, . . . , h_{N} 1110, a sequentiality of the input datasets can be leveraged.

In a single step of the processing, the recurrent machine learning model F 1112 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 1112 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 1112 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F(h)(xₙ, F^{(h)}(xₙ₋₁, hₙ₋₂)).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-4. Certain steps or functions of the methods and workflows described herein, including one or more of the steps or functions of FIGS. 1-4, may be performed by a server or by another processor in a network-based cloud-computing system. Certain steps or functions of the methods and workflows described herein, including one or more of the steps of FIGS. 1-4, may be performed by a client computer in a network-based cloud computing system. The steps or functions of the methods and workflows described herein, including one or more of the steps of FIGS. 1-4, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-4, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1202 that may be used to implement systems, apparatuses, and methods described herein is depicted in FIG. 12. Computer 1202 includes a processor 1204 operatively coupled to a data storage device 1212 and a memory 1210. Processor 1204 controls the overall operation of computer 1202 by executing computer program instructions that define such operations. The computer program instructions may be stored in data storage device 1212, or other computer readable medium, and loaded into memory 1210 when execution of the computer program instructions is desired. Thus, the method and workflow steps or functions of FIGS. 1-4 can be defined by the computer program instructions stored in memory 1210 and/or data storage device 1212 and controlled by processor 1204 executing the computer program instructions. For example, the computer program instructions can be implemented as computer executable code programmed by one skilled in the art to perform the method and workflow steps or functions of FIGS. 1-4. Accordingly, by executing the computer program instructions, the processor 1204 executes the method and workflow steps or functions of FIGS. 1-4. Computer 1202 may also include one or more network interfaces 1206 for communicating with other devices via a network. Computer 1202 may also include one or more input/output devices 1208 that enable user interaction with computer 1202 (e.g., display, keyboard, mouse, speakers, buttons, etc.).

Processor 1204 may include both general and special purpose microprocessors, and may be the sole processor or one of multiple processors of computer 1202. Processor 1204 may include one or more central processing units (CPUs), for example. Processor 1204, data storage device 1212, and/or memory 1210 may include, be supplemented by, or incorporated in, one or more application-specific integrated circuits (ASICs) and/or one or more field programmable gate arrays (FPGAs).

Data storage device 1212 and memory 1210 each include a tangible non-transitory computer readable storage medium. Data storage device 1212, and memory 1210, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1208 may include peripherals, such as a printer, scanner, display screen, etc. For example, input/output devices 1208 may include a display device such as a cathode ray tube (CRT) or liquid crystal display (LCD) monitor for displaying information to the user, a keyboard, and a pointing device such as a mouse or a trackball by which the user can provide input to computer 1202.

An image acquisition device 1214 can be connected to the computer 1202 to input image data (e.g., medical images) to the computer 1202. It is possible to implement the image acquisition device 1214 and the computer 1202 as one device. It is also possible that the image acquisition device 1214 and the computer 1202 communicate wirelessly through a network. In a possible embodiment, the computer 1202 can be located remotely with respect to the image acquisition device 1214.

Any or all of the systems, apparatuses, and methods discussed herein may be implemented using one or more computers such as computer 1202.

One skilled in the art will recognize that an implementation of an actual computer or computer system may have other structures and may contain other components as well, and that FIG. 12 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) one or more input medical images each in a domain and 2) a domain code for each of the one or more input medical images identifying its domain; for each respective one of the domain codes, determining one or more weights based on the respective domain code; updating one or more parameters of a dynamic convolutional layer based on the one or more weights; extracting a first set of features from the one or more input medical images using the dynamic convolutional layer with the one or more updated parameters; encoding the first set of features into a second set of features using a machine learning based encoder; performing a medical imaging analysis task based on the second set of features; and outputting results of the medical imaging analysis task.

Illustrative embodiment 2. The computer-implemented method of illustrative embodiment 1, wherein the dynamic convolutional layer and the machine learning based encoder are trained by: receiving 1) one or more training medical images each in a domain and 2) a training domain code for each of the one or more training medical images identifying its domain; masking patches of the one or more training medical images; for each respective one of the training domain codes, determining one or more additional weights based on the respective training domain code; updating one or more additional parameters of the dynamic convolutional layer based on the one or more additional weights; extracting a third set of features from the unmasked patches of the one or more training medical images using the dynamic convolutional layer with the one or more updated additional parameters; encoding the third set of features into a fourth set of features using the machine learning based encoder; inserting tokens representing the masked patches of the one or more training medical images into the fourth set of features; reconstructing the one or more training medical images based on the fourth set of features with the inserted tokens using a machine learning based decoder; training the dynamic convolutional layer and the machine learning based encoder based on the one or more training medical images and the one or more reconstructed training medical images; and outputting the trained dynamic convolutional layer and the trained machine learning based encoder.

Illustrative embodiment 3. The computer-implemented method of any one of illustrative embodiments 1-2, wherein determining one or more weights based on the respective domain code comprises: projecting the respective domain code to the one or more weights using a linear projector.

Illustrative embodiment 4. The computer-implemented method of any one of illustrative embodiments 1-3, wherein updating one or more parameters of a dynamic convolutional layer based on the one or more weights comprises: updating a weight parameter and a bias parameter of the dynamic convolutional layer.

Illustrative embodiment 5. The computer-implemented method of any one of illustrative embodiments 1-4, wherein updating one or more parameters of a dynamic convolutional layer based on the one or more weights comprises: determining a dot product of the one or more parameters of the dynamic convolutional layer and a respective one of the one or more weights.

Illustrative embodiment 6. The computer-implemented method of any one of illustrative embodiments 1-5, wherein encoding the first set of features into a second set of features using a machine learning based encoder comprises: encoding the first set of features with positional embeddings.

Illustrative embodiment 7. The computer-implemented method of any one of illustrative embodiments 1-6, wherein encoding the first set of features into a second set of features using a machine learning based encoder comprises: generating modality embeddings based on the domain codes; and encoding the first set of features with the modality embeddings.

Illustrative embodiment 8. The computer-implemented method of any one of illustrative embodiments 1-7, wherein the medical imaging analysis task comprises medical image synthesis.

Illustrative embodiment 9. An apparatus comprising: means for receiving 1) one or more input medical images each in a domain and 2) a domain code for each of the one or more input medical images identifying its domain; for each respective one of the domain codes, means for determining one or more weights based on the respective domain code; means for updating one or more parameters of a dynamic convolutional layer based on the one or more weights; means for extracting a first set of features from the one or more input medical images using the dynamic convolutional layer with the one or more updated parameters; means for encoding the first set of features into a second set of features using a machine learning based encoder; means for performing a medical imaging analysis task based on the second set of features; and means for outputting results of the medical imaging analysis task.

Illustrative embodiment 10. The apparatus of illustrative embodiment 9, wherein the dynamic convolutional layer and the machine learning based encoder are trained by: receiving 1) one or more training medical images each in a domain and 2) a training domain code for each of the one or more training medical images identifying its domain; masking patches of the one or more training medical images; for each respective one of the training domain codes, determining one or more additional weights based on the respective training domain code; updating one or more additional parameters of the dynamic convolutional layer based on the one or more additional weights; extracting a third set of features from the unmasked patches of the one or more training medical images using the dynamic convolutional layer with the one or more updated additional parameters; encoding the third set of features into a fourth set of features using the machine learning based encoder; inserting tokens representing the masked patches of the one or more training medical images into the fourth set of features; reconstructing the one or more training medical images based on the fourth set of features with the inserted tokens using a machine learning based decoder; training the dynamic convolutional layer and the machine learning based encoder based on the one or more training medical images and the one or more reconstructed training medical images; and outputting the trained dynamic convolutional layer and the trained machine learning based encoder.

Illustrative embodiment 11. The apparatus of any one of illustrative embodiments 9-10, wherein the means for determining one or more weights based on the respective domain code comprises: means for projecting the respective domain code to the one or more weights using a linear projector.

Illustrative embodiment 12. The apparatus of any one of illustrative embodiments 9-11, wherein the means for updating one or more parameters of a dynamic convolutional layer based on the one or more weights comprises: means for updating a weight parameter and a bias parameter of the dynamic convolutional layer.

Illustrative embodiment 13. The apparatus of any one of illustrative embodiments 9-12, wherein the means for updating one or more parameters of a dynamic convolutional layer based on the one or more weights comprises: means for determining a dot product of the one or more parameters of the dynamic convolutional layer and a respective one of the one or more weights.

Illustrative embodiment 14. A non-transitory computer-readable storage medium comprising instructions which, when executed by a computer, cause the computer to carry out operations comprising: receiving 1) one or more input medical images each in a domain and 2) a domain code for each of the one or more input medical images identifying its domain; for each respective one of the domain codes, determining one or more weights based on the respective domain code; updating one or more parameters of a dynamic convolutional layer based on the one or more weights; extracting a first set of features from the one or more input medical images using the dynamic convolutional layer with the one or more updated parameters; encoding the first set of features into a second set of features using a machine learning based encoder; performing a medical imaging analysis task based on the second set of features; and outputting results of the medical imaging analysis task.

Illustrative embodiment 15. The non-transitory computer-readable storage medium of illustrative embodiment 14, wherein the dynamic convolutional layer and the machine learning based encoder are trained by: receiving 1) one or more training medical images each in a domain and 2) a training domain code for each of the one or more training medical images identifying its domain; masking patches of the one or more training medical images; for each respective one of the training domain codes, determining one or more additional weights based on the respective training domain code; updating one or more additional parameters of the dynamic convolutional layer based on the one or more additional weights; extracting a third set of features from the unmasked patches of the one or more training medical images using the dynamic convolutional layer with the one or more updated additional parameters; encoding the third set of features into a fourth set of features using the machine learning based encoder; inserting tokens representing the masked patches of the one or more training medical images into the fourth set of features; reconstructing the one or more training medical images based on the fourth set of features with the inserted tokens using a machine learning based decoder; training the dynamic convolutional layer and the machine learning based encoder based on the one or more training medical images and the one or more reconstructed training medical images; and outputting the trained dynamic convolutional layer and the trained machine learning based encoder.

Illustrative embodiment 16. The non-transitory computer-readable storage medium of any one of illustrative embodiments 14-15, wherein encoding the first set of features into a second set of features using a machine learning based encoder comprises: encoding the first set of features with positional embeddings.

Illustrative embodiment 17. The non-transitory computer-readable storage medium of any one of illustrative embodiments 14-16, wherein encoding the first set of features into a second set of features using a machine learning based encoder comprises: generating modality embeddings based on the domain codes; and encoding the first set of features with the modality embeddings.

Illustrative embodiment 18. The non-transitory computer-readable storage medium of any one of illustrative embodiments 14-17, wherein the medical imaging analysis task comprises medical image synthesis.

Illustrative embodiment 19. A computer-implemented method comprising: receiving 1) one or more training medical images each in a domain and 2) a domain code for each of the one or more training medical images identifying its domain; masking patches of the one or more training medical images; for each respective one of the domain codes, determining one or more weights based on the respective domain code; updating one or more parameters of a dynamic convolutional layer based on the one or more additional weights; extracting a first set of features from the unmasked patches of the one or more training medical images using the dynamic convolutional layer with the one or more updated parameters; encoding the first set of features into a second set of features using a machine learning based encoder; inserting tokens representing the masked patches of the one or more training medical images into the second set of features; reconstructing the one or more training medical images based on the second set of features with the inserted tokens using a machine learning based decoder; training the dynamic convolutional layer and the machine learning based encoder based on the one or more training medical images and the one or more reconstructed training medical images; and outputting the trained dynamic convolutional layer and the trained machine learning based encoder.

Illustrative embodiment 20. The computer-implemented method of illustrative embodiment 19, wherein determining one or more weights based on the respective domain code comprises: projecting the respective domain code to the one or more weights using a linear projector.

1. A computer-implemented method comprising:
receiving 1) one or more input medical images each in a domain and 2) a domain code for each of the one or more input medical images identifying its domain;
for each respective one of the domain codes, determining one or more weights based on the respective domain code;
updating one or more parameters of a dynamic convolutional layer based on the one or more weights;
extracting a first set of features from the one or more input medical images using the dynamic convolutional layer with the one or more updated parameters;
encoding the first set of features into a second set of features using a machine learning based encoder;
performing a medical imaging analysis task based on the second set of features; and
outputting results of the medical imaging analysis task.

2. The computer-implemented method of claim 1, wherein the dynamic convolutional layer and the machine learning based encoder are trained by:
receiving 1) one or more training medical images each in a domain and 2) a training domain code for each of the one or more training medical images identifying its domain;
masking patches of the one or more training medical images;
for each respective one of the training domain codes, determining one or more additional weights based on the respective training domain code;
updating one or more additional parameters of the dynamic convolutional layer based on the one or more additional weights;
extracting a third set of features from the unmasked patches of the one or more training medical images using the dynamic convolutional layer with the one or more updated additional parameters;
encoding the third set of features into a fourth set of features using the machine learning based encoder;
inserting tokens representing the masked patches of the one or more training medical images into the fourth set of features;
reconstructing the one or more training medical images based on the fourth set of features with the inserted tokens using a machine learning based decoder;
training the dynamic convolutional layer and the machine learning based encoder based on the one or more training medical images and the one or more reconstructed training medical images; and
outputting the trained dynamic convolutional layer and the trained machine learning based encoder.

3. The computer-implemented method of claim 1, wherein determining one or more weights based on the respective domain code comprises:
projecting the respective domain code to the one or more weights using a linear projector.

4. The computer-implemented method of claim 1, wherein updating one or more parameters of a dynamic convolutional layer based on the one or more weights comprises:
updating a weight parameter and a bias parameter of the dynamic convolutional layer.

5. The computer-implemented method of claim 1, wherein updating one or more parameters of a dynamic convolutional layer based on the one or more weights comprises:
determining a dot product of the one or more parameters of the dynamic convolutional layer and a respective one of the one or more weights.

6. The computer-implemented method of claim 1, wherein encoding the first set of features into a second set of features using a machine learning based encoder comprises:
encoding the first set of features with positional embeddings.

7. The computer-implemented method of claim 1, wherein encoding the first set of features into a second set of features using a machine learning based encoder comprises:
generating modality embeddings based on the domain codes; and
encoding the first set of features with the modality embeddings.

8. The computer-implemented method of claim 1, wherein the medical imaging analysis task comprises medical image synthesis.

9. An apparatus comprising:
means for receiving 1) one or more input medical images each in a domain and 2) a domain code for each of the one or more input medical images identifying its domain;
for each respective one of the domain codes, means for determining one or more weights based on the respective domain code;
means for updating one or more parameters of a dynamic convolutional layer based on the one or more weights;
means for extracting a first set of features from the one or more input medical images using the dynamic convolutional layer with the one or more updated parameters;
means for encoding the first set of features into a second set of features using a machine learning based encoder;
means for performing a medical imaging analysis task based on the second set of features; and
means for outputting results of the medical imaging analysis task.

10. The apparatus of claim 9, wherein the dynamic convolutional layer and the machine learning based encoder are trained by:
receiving 1) one or more training medical images each in a domain and 2) a training domain code for each of the one or more training medical images identifying its domain;
masking patches of the one or more training medical images;
for each respective one of the training domain codes, determining one or more additional weights based on the respective training domain code;
updating one or more additional parameters of the dynamic convolutional layer based on the one or more additional weights;
extracting a third set of features from the unmasked patches of the one or more training medical images using the dynamic convolutional layer with the one or more updated additional parameters;
encoding the third set of features into a fourth set of features using the machine learning based encoder;
inserting tokens representing the masked patches of the one or more training medical images into the fourth set of features;
reconstructing the one or more training medical images based on the fourth set of features with the inserted tokens using a machine learning based decoder;
training the dynamic convolutional layer and the machine learning based encoder based on the one or more training medical images and the one or more reconstructed training medical images; and
outputting the trained dynamic convolutional layer and the trained machine learning based encoder.

11. The apparatus of claim 9, wherein the means for determining one or more weights based on the respective domain code comprises:
means for projecting the respective domain code to the one or more weights using a linear projector.

12. The apparatus of claim 9, wherein the means for updating one or more parameters of a dynamic convolutional layer based on the one or more weights comprises:
means for updating a weight parameter and a bias parameter of the dynamic convolutional layer.

13. The apparatus of claim 9, wherein the means for updating one or more parameters of a dynamic convolutional layer based on the one or more weights comprises:
means for determining a dot product of the one or more parameters of the dynamic convolutional layer and a respective one of the one or more weights.

14. A non-transitory computer-readable storage medium comprising instructions which, when executed by a computer, cause the computer to carry out operations comprising:
receiving 1) one or more input medical images each in a domain and 2) a domain code for each of the one or more input medical images identifying its domain;
for each respective one of the domain codes, determining one or more weights based on the respective domain code;
updating one or more parameters of a dynamic convolutional layer based on the one or more weights;
extracting a first set of features from the one or more input medical images using the dynamic convolutional layer with the one or more updated parameters;
encoding the first set of features into a second set of features using a machine learning based encoder;
performing a medical imaging analysis task based on the second set of features; and
outputting results of the medical imaging analysis task.

15. The non-transitory computer-readable storage medium of claim 14, wherein the dynamic convolutional layer and the machine learning based encoder are trained by:
receiving 1) one or more training medical images each in a domain and 2) a training domain code for each of the one or more training medical images identifying its domain;
masking patches of the one or more training medical images;
for each respective one of the training domain codes, determining one or more additional weights based on the respective training domain code;
updating one or more additional parameters of the dynamic convolutional layer based on the one or more additional weights;
extracting a third set of features from the unmasked patches of the one or more training medical images using the dynamic convolutional layer with the one or more updated additional parameters;
encoding the third set of features into a fourth set of features using the machine learning based encoder;
inserting tokens representing the masked patches of the one or more training medical images into the fourth set of features;
reconstructing the one or more training medical images based on the fourth set of features with the inserted tokens using a machine learning based decoder;
training the dynamic convolutional layer and the machine learning based encoder based on the one or more training medical images and the one or more reconstructed training medical images; and
outputting the trained dynamic convolutional layer and the trained machine learning based encoder.

16. The non-transitory computer-readable storage medium of claim 14, wherein encoding the first set of features into a second set of features using a machine learning based encoder comprises:
encoding the first set of features with positional embeddings.

17. The non-transitory computer-readable storage medium of claim 14, wherein encoding the first set of features into a second set of features using a machine learning based encoder comprises:
generating modality embeddings based on the domain codes; and
encoding the first set of features with the modality embeddings.

18. The non-transitory computer-readable storage medium of claim 14, wherein the medical imaging analysis task comprises medical image synthesis.

19. A computer-implemented method comprising:
receiving 1) one or more training medical images each in a domain and 2) a domain code for each of the one or more training medical images identifying its domain;
masking patches of the one or more training medical images;
for each respective one of the domain codes, determining one or more weights based on the respective domain code;
updating one or more parameters of a dynamic convolutional layer based on the one or more additional weights;
extracting a first set of features from the unmasked patches of the one or more training medical images using the dynamic convolutional layer with the one or more updated parameters;
encoding the first set of features into a second set of features using a machine learning based encoder;
inserting tokens representing the masked patches of the one or more training medical images into the second set of features;
reconstructing the one or more training medical images based on the second set of features with the inserted tokens using a machine learning based decoder;
training the dynamic convolutional layer and the machine learning based encoder based on the one or more training medical images and the one or more reconstructed training medical images; and
outputting the trained dynamic convolutional layer and the trained machine learning based encoder.

20. The computer-implemented method of claim 19, wherein determining one or more weights based on the respective domain code comprises:
projecting the respective domain code to the one or more weights using a linear projector.
